(19) United States
(12) Patent Application Publication
(10) Pub. No.: US 2015/0335389 A1
(43) Pub. Date: Nov. 26, 2015

(54) SURGICAL TENSION SETTING SYSTEM

(71) Applicant: I. Melbourne GREENBERG, Roslyn Estates, NY (US)

(72) Inventor: I. Melbourne GREENBERG, Roslyn Estates, NY (US)

(21) Appl. No.: 14/282,586

(22) Filed: May 20, 2014

(51) Int. Cl. A61B 19/00

(52) U.S. Cl. CPC A61B 19/26

(57) ABSTRACT

An implement holding device for use in surgeries is disclosed. The implement holding device includes a flexible arm that defines an inner lumen and that is configured to be coupled to a medical instrument. A tension cable is disposed within the inner lumen and is configured to adjust the rigidity of the flexible arm. Additionally, a tension setting dial is coupled to the tension cable. The tension setting dial includes a plurality of discrete tension setting locators, each locator is configured to be engaged when the tension setting dial is in a position corresponding to the locator, and each locator corresponds to a different cable tension force such that manipulating the tension setting dial to a position corresponding to one of the locators adjusts the cable tension to a specific cable tension force, and wherein changing the dial position is effective to change the rigidity of the flexible arm.

### TECHNICAL FIELD

This disclosure is generally directed to a tension setting system, and more particularly, to a surgical tension setting system for use in surgical procedures.

### BACKGROUND

During surgical procedures, such as neurosurgery, a surgeon typically uses optical magnification to view the surgical site. For example, an operating microscope may be used or an endoscope can be inserted into the patient to visualize the anatomy and pathology such as a tumor. The endoscope relays images to a monitor, which displays magnified real-time video from the endoscope. The surgeon may perform at least a portion of the surgery while looking exclusively at the monitor. The use of an optical magnification system allows the surgeon to reach the target location while minimizing trauma to the surrounding tissue. Optical magnification systems are particularly useful in neurosurgical operations. Exemplary neurosurgical procedures include, for example, removing a tumor, decompressing a cranial nerve, and taking a biopsy sample. Additional procedures may include spinal operations, for example, removing a herniated disc.

When using a magnification system, the surgeon's line of sight is typically focused straight ahead on the monitor or in the binocular of the operating microscope while the surgeon manipulates surgical instruments at the operating site located below. Surgical procedures may last for many hours, thus requiring the surgeon to be focused on the monitor or binocular of the operating microscope for long spans of time. Surgeons often suffer from fatigue due to the endurance required to be focused for such long surgical procedures. There is, therefore, a great need to reduce any unnecessary movements or actions by the surgeon that superfluously delay the course of the surgery. In particular, any movement of the surgeon requiring the surgeon's line of sight to be removed from the binocular of the operating microscope or monitor will delay the surgery. For example, when the surgeon removes his eyesight from the binocular of the operating microscope or the monitor to pick up a surgical instrument, or moves the location of a single surgical instrument, the surgery is delayed because the surgeon needs to readjust his eyes each time he returns to focus on the operative field. Many hospitals require several nurses to retrieve and move the surgical instruments for the surgeon so that the surgeon can maintain his eyesight through the binocular of the operating microscope or on the monitor. Of course, additional nurses impose increased costs on the patient.

In some procedures, surgeons are required to stand in a non-ergonomic position for many hours. For example, in order to properly reach a surgical site, the surgeon might need to position his arms extending slightly forward and away from his body such that his elbows are bent at an angle. Again, the surgeon's head, and thus his line of sight, is directed straight ahead toward a binocular of the operating microscope or monitor. This non-ergonomic position strains the surgeon's back and arms after several hours, and causes fatigue.

A rigid framework has been used to ameliorate surgeon fatigue. The framework is positioned above the surgical site, and includes attachments for various surgical instruments. A flexible arm typically connects each surgical instrument to the rigid framework. This arrangement can decrease superfluous movements of the surgeon, and thus reduce the fatigue associated with such long surgical procedures. For example, the framework allows the surgical instruments to be positioned at an easy to grasp location for the surgeon which is adjacent to the surgical site but remains in the operative field. Therefore, the surgeon is not required to remove his eyesight from the operating microscope or the monitor to move from one instrument to another instrument or to move the location of a single instrument. This also results in a decreased number of nurses required to perform the surgical procedure, thus saving operating time and overall costs for the procedure.

Cables within each flexible arm allow the tension of the flexible arm to be adjusted. A lower or softer tension may allow the flexible arm, and thus the surgical instrument, to move more easily. During the surgical procedure, the surgeon often requires a specific tension for each instrument attached to the framework, and the tension requirement changes depending on the surgical task. For example, when retracting tissue, the surgeon may require a higher tension setting on a retractor blade so that the retractor blade moves very little yet is not so tight as to cause damage to the retracted tissue. Use of the flexible arm with a retractor blade requires very subtle movements and tension on the flexible arm. Another example is when a surgeon is dissecting tissue. This allows the surgeon to carefully dissect tissue around delicate nerves in the brain with the retractor blade. However, a surgical drill may require different movements, and therefore the surgeon may use a lower tension setting for the drilling instrument.

This disclosure provides an implement holding device including a tension setting system that allows a surgeon to easily adjust the tension for specific medical instruments, and for different uses associated with each instrument, while maintaining his focus on the binocular of the operating microscope or a monitor.

### SUMMARY

The present disclosure is directed to an implement holding device for use in surgeries. The implement holding device may include a flexible arm defining an inner lumen and having a portion that is configured to be coupled to a medical instrument. A tension cable may be disposed within the inner lumen and may be configured to adjust the rigidity of the flexible arm. Additionally, a tension setting dial may be coupled to the tension cable and may be configured to be manipulated to adjust the tension of the tension cable. The tension setting dial may include a plurality of discrete tension setting locators, each tension setting locator may be configured to be engaged when the tension setting dial is in a position corresponding to the tension setting locator, and each tension setting locator may correspond to a different cable tension force such that manipulating the tension setting dial to a position corresponding to one of the tension setting locators adjusts the cable tension to a specific cable tension force, and wherein changing the dial position is effective to change the rigidity of the flexible arm.

The present disclosure is directed toward a method of adjusting tension in an implement holding device. The method may include rotating the tension setting dial from a first locator to a second locator to adjust the cable tension and manipulating the position of the flexible arm. Additionally, the method may include applying a downward force on the flexible arm such that the flexible arm provides sufficient tension and pressure against the downward force.

The present disclosure is directed toward a tension setting quantification system including a load cell and an output display. The load cell may be coupled to the tension cable of an implement holding device such that the load cell is configured to convert the tension in the tension cable into a signal. The output display may be configured to display the signal to a user.

The present disclosure is directed toward a method of quantifying tension in a tension setting quantification system. The method may include adjusting the tension in the tension cable by rotating the tension setting dial to a tension setting locator and adjusting the length of the tension cable if the output display produces a signal that does not correspond to a predetermined cable tension associated with the tension setting locator.

### BRIEF DESCRIPTION OF THE DRAWINGS

FIG. 1 is a schematic illustration of a rigid framework system for a surgical procedure;

FIG. 2A is another schematic illustration of the rigid framework system;

FIGS. 2B and 2C are schematic illustrations of the rigid framework system during a surgical procedure;

FIG. 3 is a schematic illustration of a prior art flexible arm used in the rigid framework system of FIGS. 1-2C;

FIG. 4 is an exemplary embodiment of a flexible arm and tension setting system of the present disclosure;

FIGS. 5A and 5B are schematic illustrations of the flexible arm of FIG. 4;

FIG. 6 is a schematic illustration of the tension setting system of FIG. 4;

FIG. 7 is a schematic illustration of an indicator of the tension setting system of FIG. 4;

FIG. 8 is another schematic illustration of an indicator of the tension setting system of FIG. 4;

FIG. 9A is a schematic illustration of a first embodiment of an attachment of the flexible arm of FIG. 4;

FIG. 9B is a schematic illustration of a second embodiment of an attachment of the flexible arm of FIG. 4;

FIG. 10 is a schematic illustration of an exemplary embodiment of the tension setting system of FIG. 4;

FIG. 11A is a schematic illustration of the tension setting system of FIG. 4 and a rigid framework system;

FIG. 11B is a schematic illustration of an exemplary embodiment of the tension setting system of claim 4;

FIG. 12 is a schematic illustration of a tension setting quantification system used with the tension setting system of FIG. 4; and

FIG. 13 is a schematic illustration of a complex connector used with the rigid framework system of FIGS. 1-2C.

### DETAILED DESCRIPTION

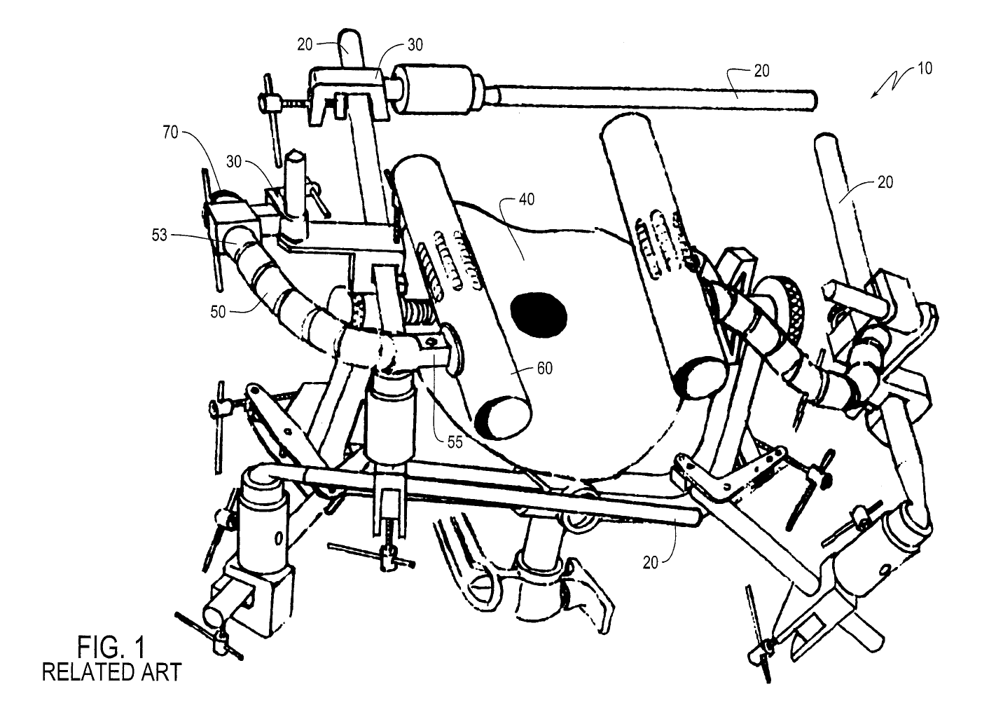

FIG. 1 illustrates a rigid framework system 10 for use in surgical operations. Support bars 20 may be coupled together with connectors 30 to form rigid framework system 10 such that rigid framework system 10 is disposed above and over surgical site 40. The framework system 10 is typically rigidly connected to the skull clamp of the patient's operating table (not pictured) or the operating table rails (not pictured). In one embodiment, surgical site 40 includes a patient's exposed brain tissue. In other embodiments, surgical site 40 may be, for example, a patient's abdominal or pelvic cavity during a laparoscopic procedure. As shown in FIG. 1, a flexible arm 50 is coupled to connector 30 for attachment to a support bar 20. A first end 53 of the flexible arm 50 is attached to connector 30 and a second end 55 of the flexible atm 50 is attached to a medical instrument.

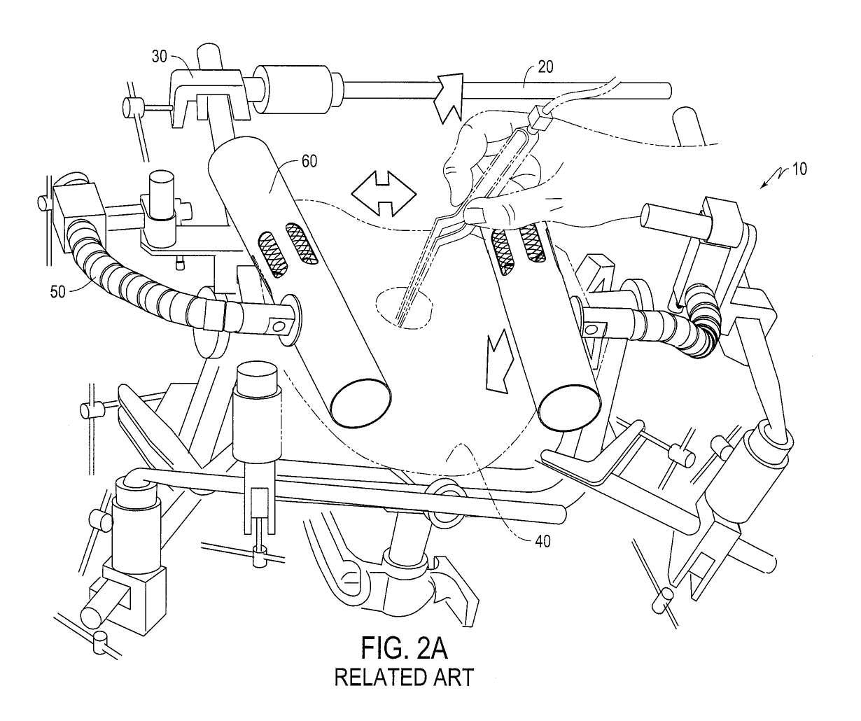

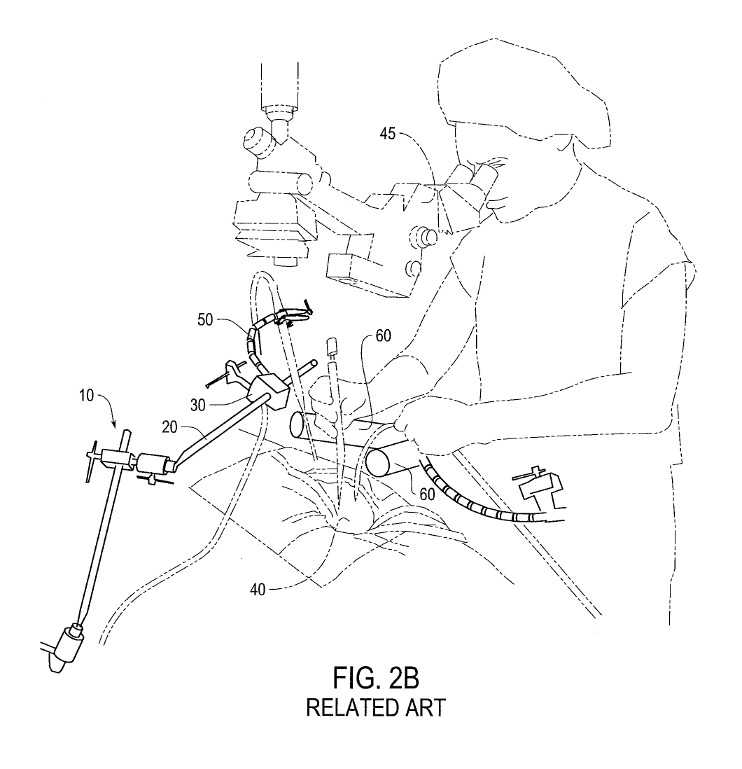

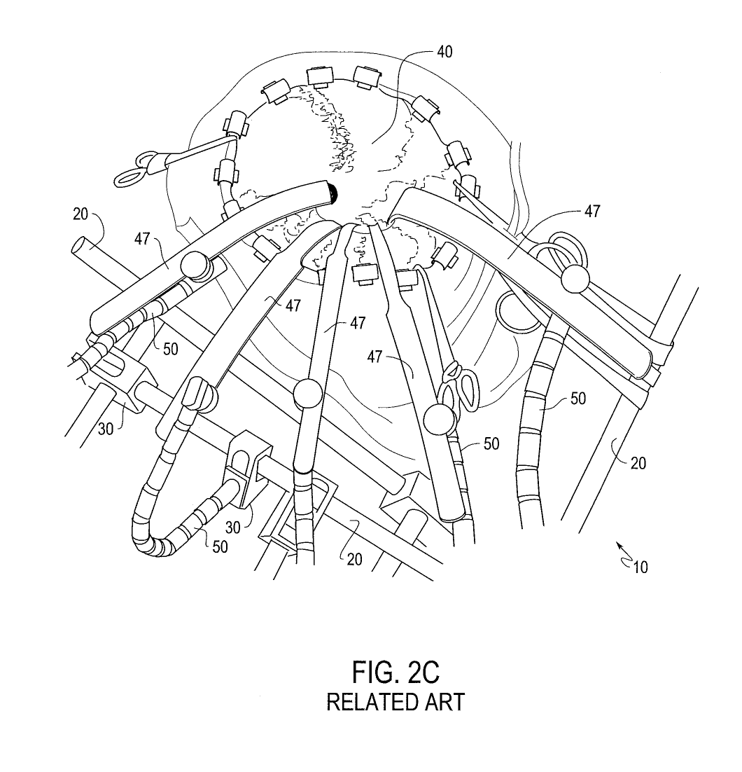

In the embodiment of FIG. 1, the medical instrument includes a hand rest 60 configured for a surgeon to rest his hand during a surgical procedure. The surgeon can rest his hand(s) and/or wrist(s) on hand rest 60 while manipulating another medical instrument within surgical site 40 (FIGS. 2B and 2B). This may allow the surgeon to manipulate the tissue within surgical site 40 while viewing the tissue through operating microscope 45 (FIG. 2B). As discussed further below, the tension in flexible arm 50 can be adjusted so that flexible arm 50 absorbs at least some of the surgeon's weight on hand rest 60. This can reduce fatigue for the surgeon during a surgical procedure. In the embodiment of FIG. 2C, multiple flexible arms 50 are connected to framework system 10 to manipulate tissue within surgical site 40. In this embodiment, for example, the flexible arms 50 may be connected to retractor blades 47 to hold open surgical site 40. The tension of each flexible arm 50 may be adjusted to provide sufficient retraction force to the tissue within surgical site 40.

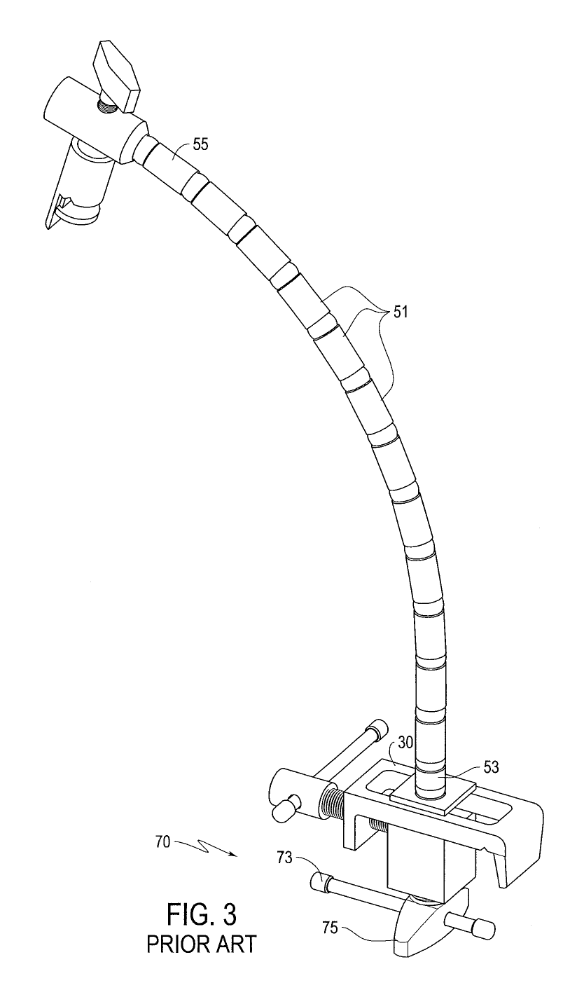

First end 53 of flexible arm 50 may also include a tension setting mechanism 70 configured to adjust the tension within flexible arm 50. As shown in FIG. 3, known tension setting mechanisms 70 include a pivot bar 73 and a rotational member 75 that is attached to cables extending within the flexible arm 50. A user can grasp and rotate pivot bar 73 to rotate rotational member 75. Movement of rotational member 75 in a first direction (e.g., clockwise direction) winds and tightens the internal cables, which increases the tension between adjacent flexible arm segments 51, and causes the rigidity of flexible arm 50 to increase. Conversely, movement of rotational member 75 in a second direction (e.g., counterclockwise) unwinds and loosens the internal cables, which decreases the tension between adjacent flexible arm segments 51, and causes the rigidity of the flexible arm 50 to decrease. Therefore, a surgeon, or nurse, may rotate pivot bar 73 during a surgical procedure to achieve the desired tension in flexible arm 50 for a specific surgical procedure.

However, tension setting system 70 of known devices requires the surgeon to find the desired setting for a specific application by trial and error, and any adjustment of the tension setting requires the surgeon to avert his line of sight from the operative field or optical magnification system (e.g., operating microscope 45) and to the pivot bar 73. For example, in order to dissect or retract delicate brain tissue with a retractor blade (e.g., retractor blade 47), the surgeon, nurse, or assistant may be required to adjust the rotation of rotational member 75 many times until the specific tension is found. Also, most surgeons arrive at the desired tension setting by “feel,” i.e., by adjusting the tension setting until the surgical equipment achieves a rigidity that “feels” correct for a specific application. This is very difficult and time consuming for many surgeons. With known devices, the surgeon must therefore adjust the tension himself because it is difficult to communicate the desired tension setting to a nurse or surgical assistant.

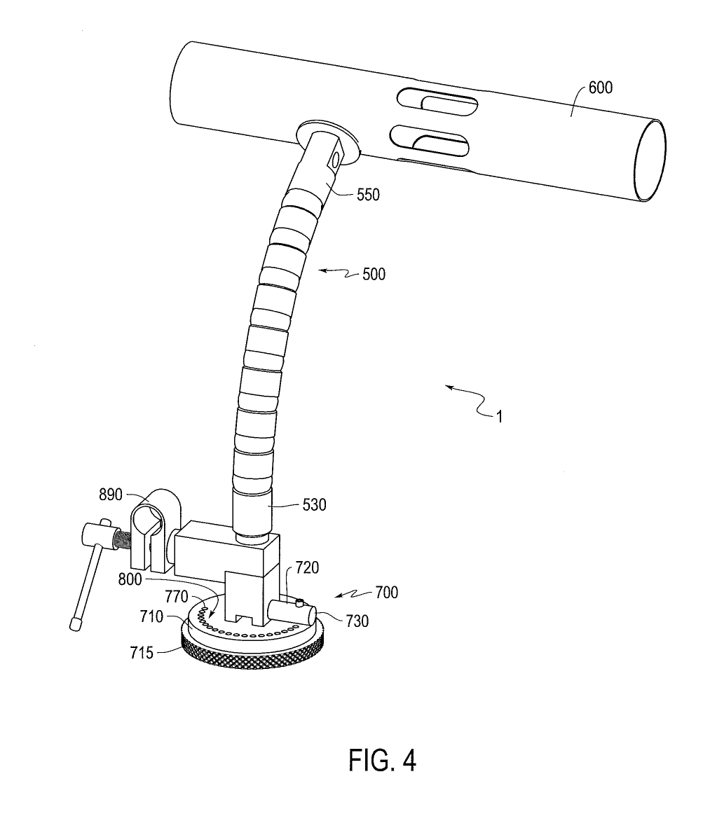

As shown in FIG. 4, embodiments of the present disclosure include an implement holding device 1 for use in surgeries comprising a flexible arm 500 having a first end 530 connected to a tension setting system 700 and a second end 550 coupled to a surgical instrument (e.g., hand rest 600, retractor blade 47). The implement holding device 1 may be connected to rigid framework system 10 by, for example, attachment member 890. The tension setting system 700 may include a tension setting dial, for example tension setting knob 710, and a position indicator 720 with an indicator end portion 730. Rotation of tension setting knob 710 with regard to indicator 720 (and thus with regard to indicator end portion 730) adjusts the tension of inner cables and thus the rigidity of the flexible arm 500. The tension setting system 700 adjusts the tension of the cables in discrete increments each corresponding to a different tension/rigidity. Additionally, tension setting knob 710 can include one or more discrete tension setting locators 800, wherein each locator 800 corresponds to a predetermined tension setting within flexible arm 500.

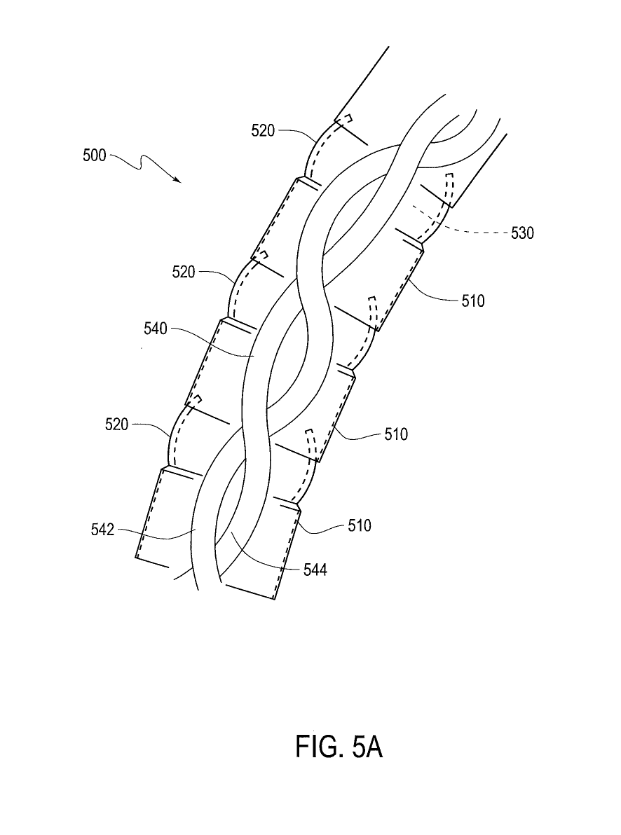

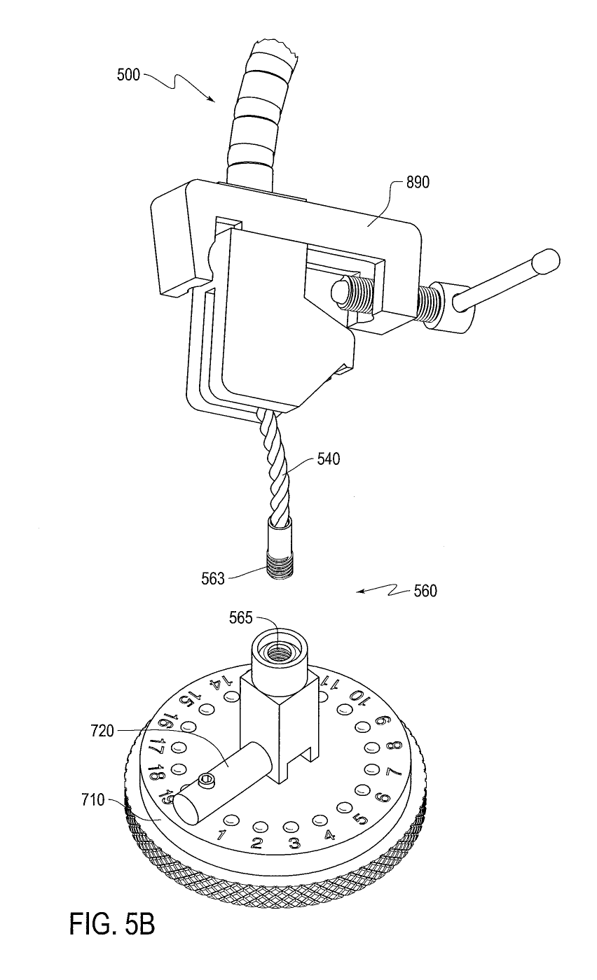

FIG. 5A represents an exemplary cross-section of flexible arm 500. A plurality of arm segments 510, each cooperating with an adjacent segment, are disposed at an outer peripheral surface of flexible arm 500, thus forming inner lumen 530. The plurality of arm segments 510 allow flexible arm 500 to bend into various configurations/shapes and to be manipulated by a user so that the user can locate the surgical tool in a desired position and orientation. For example, the arm segments 510 allow flexible arm 500 to assume an S-shape or a C-shape. As also shown in FIG. 5A, adjacent segments of the plurality of arm segments 510 may be coupled together and separated from each other by a plurality of ball joints 520. When flexible arm 500 is bent, a segment 510 along the bend may at least partly overlap a ball joint 520 to provide sufficient bending of flexible arm 500. The plurality of segments 510 and the plurality of ball joints 520 may be comprised of for example, stainless steel, titanium, Stellite®, carbon fiber materials, composites, and heat resistant plastics. Alternatively, it is further contemplated that the outer peripheral surface of flexible arm 500 may include one unitary and flexible member that is configured to bend.

The flexible arm 500 may further include one or more tension cables 540 within inner lumen 530. As shown in FIG. 5A, the tension cables 540 may include, for example, a first tension cable 542 and a second tension cable 544. The tension cables 540 may be coupled to tension setting knob 710 such that rotation of tension setting knob 710 in a first direction (e.g., clockwise) may cause the first tension cable 542 and the second tension cable 544 to twist tighter within flexible arm 500, thus increasing the tension between adjacent segments 510 of flexible arm 500 and providing flexible arm 500 with increased rigidity. When the flexible arm 500 is relatively more rigid, greater force is required to move the flexible arm. Conversely, rotation of the tension setting knob 710 in a second direction (e.g., counterclockwise) may cause the first tension cable 542 and the second tension cable 544 to at least partly unwind, thus decreasing the tension of the cables 540 and rendering the flexible arm 500 less rigid. It is further contemplated that one, three, four, six, eight, etc. tension cables 540 may be used to adjust the tension in flexible arm 500. For example, a single tension cable 540 may be disposed within inner lumen 530 such that rotation of tension setting knob 710 in the first direction (e.g., clockwise) may cause the single tension cable to twist and thus increase the tension within flexible arm 500.

An increased tension within flexible arm 500 may reduce the rigidity and movability of flexible arm 500 as compared to a decreased tension within flexible arm 500. For example, a decreased tension allows the plurality of segments 510 to more easily bend such that flexible arm 500 can be manipulated by a user. However, an increased tension causes the plurality of segments 510 to be more rigid, such that it is harder to bend and move flexible arm 500. Furthermore, an increased tension provides less “give” when a surgeon applies weight to the surgical instrument, for example, when the surgeon rests his hand and/or wrist on hand rest 600.

The one or more tension cables 540 may each include several filaments and/or strings. The filaments and/or strings can be attached by an adhesive material in order to form a unitary cable. Alternatively, the tension cables 540 may include, for example strands, braids or twisted pairs in materials such as stainless steel, titanium, Stellite®, composites or other materials or metals commonly used in the construction of cables.

As shown in FIG. 5B, the one or more tension cables 540 may be attached to tension setting knob 710 through, for example, connecting member 560. In the embodiment of FIG. 5B, connecting member 560 includes first connecting member 563 attached to the tension cables 540 and second connecting member 565 attached to the tension setting knob 710. The second connecting member 565 may be directly attached to indictor 720. In embodiments, first connecting member 563 may be screwed within and into second connecting member 565 to provide a secure attachment between tension cables 540 and tension setting knob 710. Additionally, this secure attachment may be removable by unscrewing first connecting member 563 from second connecting member 565.

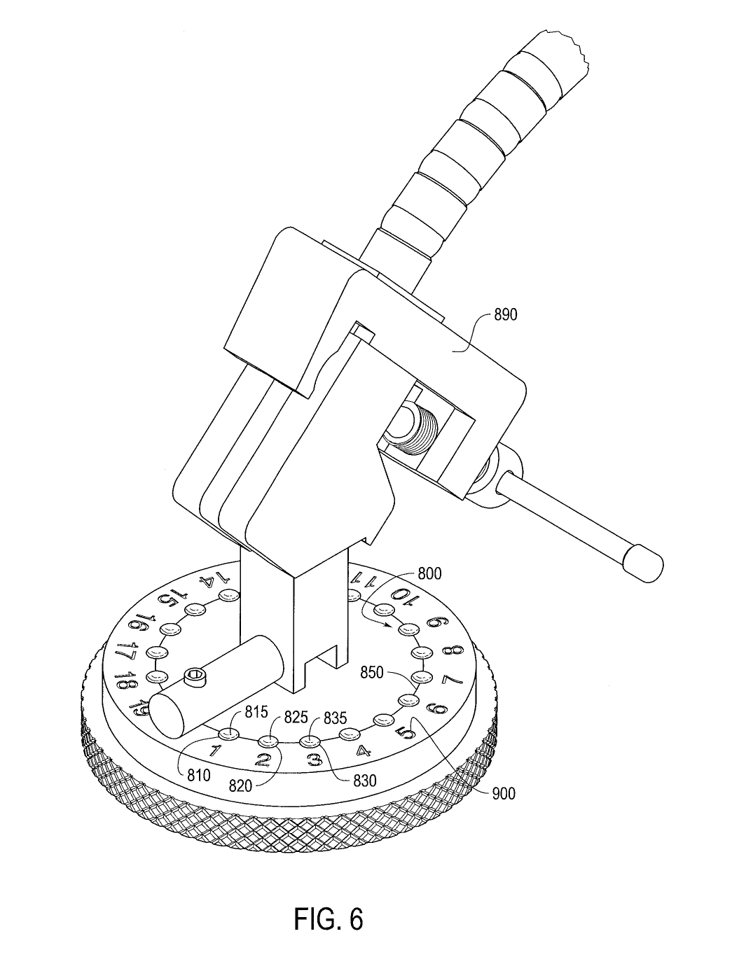

The tension setting locators 800 on tension setting knob 710 each correspond to a predetermined tension of the tension cable 540. For example, locators 800 may include at least a first locator 810, a second locator 820, and a third locator 830 (FIG. 6). Therefore, rotation of tension setting knob 710 to the first locator 810 may cause the tension cables 540 to twist a first specific amount such that tension of the tension cables 540 is a first predetermined amount. Rotation of tension setting knob 710 to the second locator 820 causes the tension cables 540 to twist a second specific amount such that tension of the tension cables 540 is a second predetermined amount. In some embodiments, the second predetermined amount may be greater than the first predetermined amount. Likewise, rotation of tension setting knob 710 to the third locator 830 causes the tension cables 540 to twist a third specific amount such that tension of the tension cables 540 is a third predetermined amount. In embodiments, the third predetermined amount is greater than both the first predetermined amount and the second predetermined amount. It is further contemplated that tension setting knob 710 may be rotated, for example, from the third locator 830 to the first locator 810, thereby causing the tension cables 540 to at least partly unwind, thus decreasing the tension of the tension cables 540. In this manner, the tension setting knob 710 is capable of setting tension in the cables 540 at a plurality of discrete predetermined settings.

As shown in FIG. 6, tension setting knob 710 may include a plurality of locators 800, for example, 10 locators, 20 locators, 30 locators, etc., wherein each locator 800 corresponds to a specific predetermined tension. The tension setting locators 800 may be circularly arranged and evenly spaced about tension setting knob 710 so that a user may easily identify each locator 800. Additionally, a unique identifying symbol and/or marker 900 may correspond to each locator 800 so that a surgeon can quickly locate the specific tension setting during a surgical procedure, and can communicate a desired tension setting to an assistant. The identifying symbols 900 may include, for example, numbers that arbitrarily represent a tension level, numbers that identify a specific tension amount, letters, colors, and/or other characters. Advantageously, each of the discrete tension settings can be pre-calibrated so that each setting corresponds to a known tension. It is very useful for the surgeon to know the amount of tension that is being applied to the surgical tool. For example, when the flexible arm 500 is coupled to a retractor blade (e.g., retractor blade 47), the surgeon will understand that certain tensions should be applied depending on the tissue that is retracted. In some embodiments, the tension setting knob 710 can be configured to have from 3 to 50 discrete tension setting positions, from 5 to 25 discrete tension setting positions, or from 10 to 20 discrete tension setting positions.

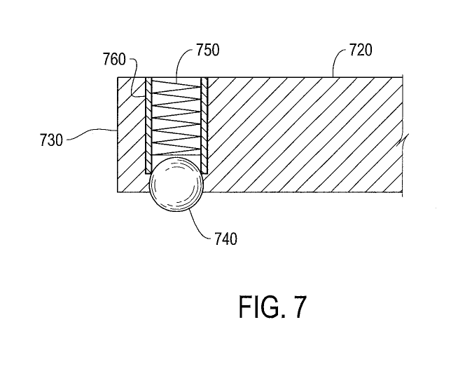

The position indicator 720 may be disposed above tension setting knob 710 such that it abuts an outer side surface of tension setting knob 710 that includes locators 800. As shown in FIG. 7, indicator end portion 730 of indicator 720 may be aligned with locators 800 in order to adjust the tension setting of flexible arm 500. In embodiments, indicator 720 includes a ball 740 that is at least partly disposed inside and at least partly disposed outside of indicator 720. A spring 750 may be configured to maintain a force on ball 740 so that ball 740 is disposed partly outside indicator 720 when in a resting position. A hollow rod 760 may be configured to hold spring 750 and ball 740 within rod 760.

In some embodiments, each tension setting can include an indent on an outer side surface of tension setting knob 710 that corresponds to the shape of ball 740. Therefore, for example, a first tension setting (e.g., at locator 810) can include a first indent 815, second tension setting (e.g., at locator 820) can include a second indent 825, and third tension setting (e.g., at locator 830) can include a third indent 835 (FIG. 6). Rotation of tension setting knob 710 so that position indicator 720 is aligned with, for example, first locator 810, may cause ball 740 to be at least partly disposed in first indent 815. The force of spring 750 urges the ball 740 into indent 815 and retains the tension setting knob 710 in the first tension setting position. Therefore, the first location 810 is engaged by indicator 720. As discussed above, this enables the tension cables 540 to be adjusted to a first predetermined tension amount. Further rotation of tension setting knob 710 so that, for example, indicator 720 moves from first locator 810 to second locator 820, causes the ball 740 to be rotated along path 850 (FIG. 6). Therefore, when ball 740 is on path 850 and is between first locator 810 and second locator 820, ball 740 may exert an upward pressure on spring 750 so that ball 740 moves upward and further into indicator 720. However, when ball 740 reaches second indent 825, because second indent 825 is disposed relatively lower than the portion of path 850 between first locator 810 and second locator 820, spring 750 forces ball 740 into second indent 825.

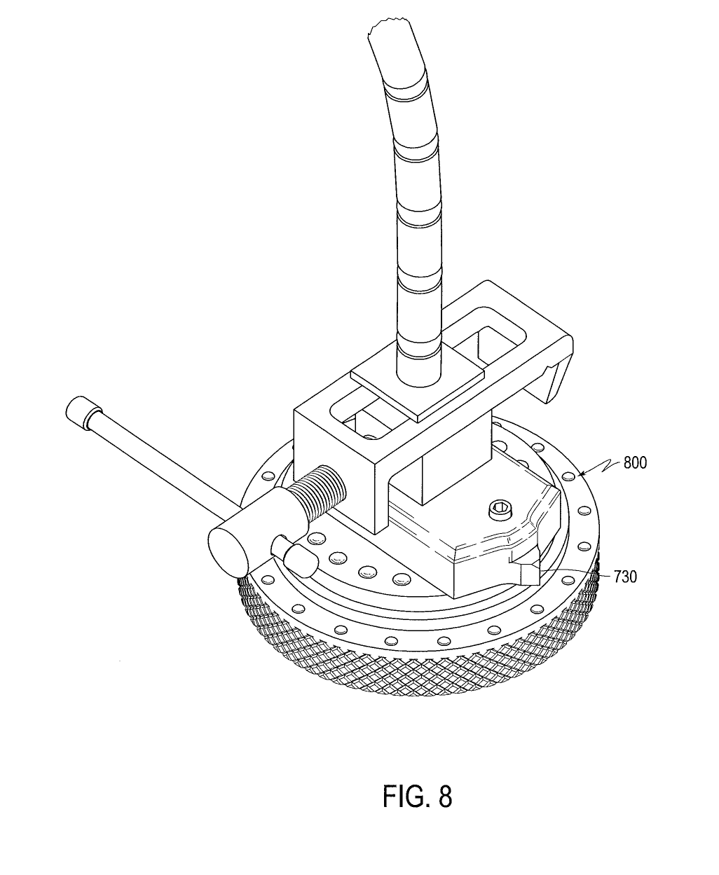

As shown in FIG. 8, indicator end portion 730 may include a point that aligns with identifying symbols 900 (and thus with locators 800). This may allow a user to easily identify which tension setting locator 800 the ball 740 is disposed within, and thus the corresponding tension on tension cables 540.

Tension setting knob 710 can be configured so that a user, for example, a surgeon or nurse, manipulates the tension setting knob 710 with his hand in order to rotate the tension setting knob 710 between, for example, first locator 810 and second locator 820. As shown in FIG. 4, tension setting knob 710 can include a knurled surface 715 on an outer perimeter of tension setting knob 710 that allows a user to easily grip and rotate tension setting knob 710. The surgeon can rotate tension setting knob 710 without moving his eyes from the binocular of the operating microscope (e.g., operating microscope 45) or the monitor during a surgical procedure, and can determine by feel the desired tension setting for the surgical task at hand, or can alternatively communicate to an assistant a desired tension setting. In embodiments, the tension setting knob 710 can be circular, with the tension setting locators 800 (e.g., indents) being positioned on a side surface of the knob in a radially outer portion of the side surface. It is further contemplated that a variety of shapes and configurations may be used for tension setting knob 710 and for the tension setting locators 800. Combining the tension setting knob 710 with several discrete tension setting locators enables the surgeon to more easily adjust the tension of the flexible arm 500 by “feel.” For example, the surgeon can rotate tension setting knob 710 with one hand without averting his sight, for example from operating microscope 45, and will be able to feel each time the tension setting system 700 is positioned at a discrete setting (e.g., because the knob 710 will weakly lock into place at each discrete tension setting when the ball 740 is pushed into the corresponding indent).

Tension setting knob 710 may be comprised of a lightweight material such that it is easy to be rotated by a user. In some embodiments, tension setting knob 710 may include, for example, a plastic material such as polytetrafluoroethylene (PTFE), Teflon®, Deirin®, Ultem® or other high temperature resistant plastic. In other embodiments, tension setting knob 710 may include stainless steel, titanium, composite materials, nickel, Stellite® alloy, or carbon fiber. It is further contemplated that tension setting knob 710 may include an outer coating, such as, for example, anodized surfaces, plating of silver, gold or other precious metals. Additionally, indicator 720 may be comprised of the same or of a different material than tension setting knob 710.

As shown in FIG. 4, tension setting knob 710 may include a stop 770 that prevents rotation of tension setting knob past a certain point. For example, when indicator 720 is aligned with the locator 800 immediately adjacent to stop 770, the user may be prevented from rotating tension setting knob 710 further in the same direction passed stop 770. Thus, the user has reached the last locator 800 in this direction and stop 770 prevents further rotation in this direction. Stop 770 therefore provides an end point to rotation of tension setting knob 710 so that the user cannot adjust the position of tension setting knob 710 beyond stop 770. It is further contemplated that tension setting knob 710 may include a first stop that prevents rotation past the last locator in the clockwise direction and a second stop that prevents rotation past the last locator in the counterclockwise direction. In other embodiments, a single stop 770 may prevent rotation past the last locator in the clockwise direction and past the last locator in the counterclockwise direction. Stop 770 may include a channel or protrusion within tension setting knob 710. In some embodiments, stop 770 may include a protrusion disposed on an outer surface of tension setting knob 710 that interacts with indictor 720 to prevent further rotation of tension setting knob 710.

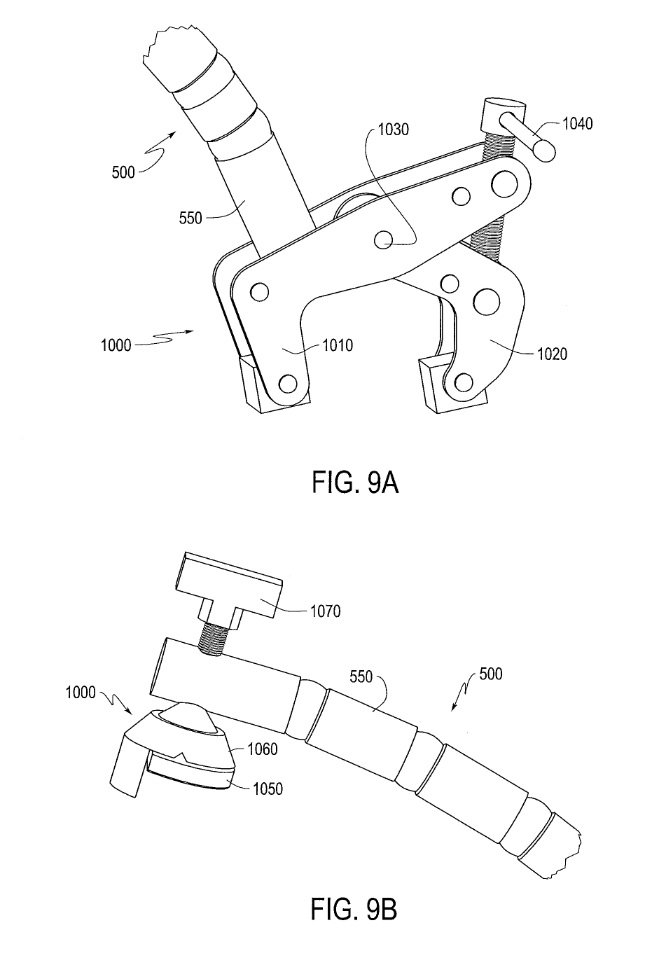

Second end 550 of flexible arm 500 may include an attachment 1000 configured for attachment to a medical instrument. As shown in FIGS. 9A and 9B, the attachment 1000 may be customized to secure a specific medical instrument to flexible arm 500. For example, the attachment 1000, as shown in FIG. 9A, may be suitable for attachment to an endoscope, a rotary cutter, a drill, an aspirator, forceps, a suction tube, and/or an ultrasonic imaging probe. The attachment 1000, as shown in FIG. 9B, may be suitable for attachment to, for example, a dissector, a small suction, a retractor blade (e.g., retractor blade 47) or any small single shaft instrument.

Attachment 1000 in FIG. 9A includes a first clamp arm 1010 and a second clamp arm 1020 configured to be pivoted toward and away from each to selectively secure a medical instrument between these components. The first clamp arm 1010 and second clamp arm 1020 may be pivoted about pivot point 1030 due to rotation of arm rotator 1040. However, it is further contemplated that other rotation mechanisms may be utilized to pivot first clamp arm 1010 and second clamp arm 1020 between open and closed states.

Attachment 1000 in FIG. 9B includes a first clamp head 1050 configured to be moved toward and away from a second clamp head 1060 to selectively secure a medical instrument between these components. The first clamp head 1050 may be moved due to rotation of arm rotator 1070. However, it is further contemplated that other rotation mechanisms may be utilized to move first clamp head 1050 between open and closed states.

Embodiments of the tension setting system 700 provide an easy to adjust system so that a user can quickly identify the desired tension setting and effortlessly obtain this setting. The user may rotate tension setting knob 710 from either, for example, first locator 810 to second locator 820, or from second locator 820 to first locator 810. Thus, the user has adjusted the tension setting of tension cables 540 and thus the tension of flexible arm 500. The user may also manipulate the position of flexible arm 500, for example by moving flexible arm 500 from a first position further from surgical site 40 to a second position closer to surgical site 40. Once in the desired position, the user may apply a downward force on flexible arm 500 such that the medical instrument is moved closer to surgical site 40. This may, for example, allow the user to manipulate tissue within surgical site 40 with the medical instrument. The flexible arm 500 can provide sufficient tension and pressure against this downward force by the user. Additionally, the user may further rotate tension setting knob 710 to third locator 830, for example, if the user determines that the specific operating procedure requires more tension in flexible arm 500.

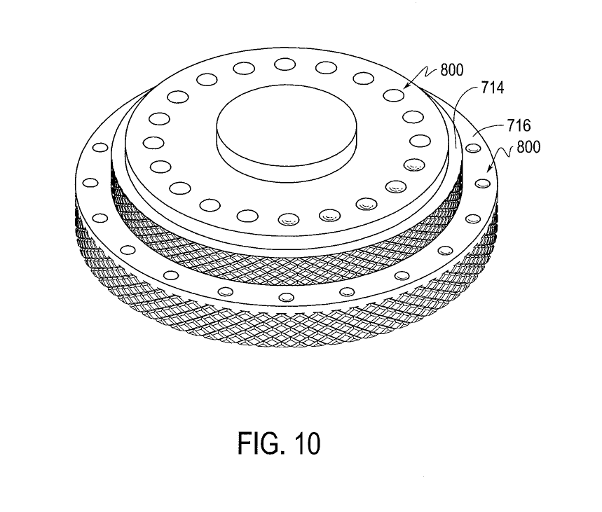

In the embodiment of FIG. 10, tension setting system 700 can include a first tension setting knob 714 and a second tension setting knob 716. The first tension setting knob 714 can be configured for coarse tension setting adjustments in tension cables 540, and the second tension setting knob 716 may be configured for fine tune adjustments in tension cables 540. Therefore, for example, the user may first adjust the tension in tension cables 540 by rotating first tension setting knob 714 to the desired locator 800. Then, the user may rotate second tension setting knob 716 to the desired locator 800 to further modify and tweak the tension of tension cables 540. The first tension setting knob 714 and second tension setting knob 716 may provide more control and flexibility for the user to obtain the desired tension in flexible arm 500. Each of the tension setting knobs can include a plurality of discrete tension settings that operate similarly to the embodiment described in connection with FIGS. 4-8.

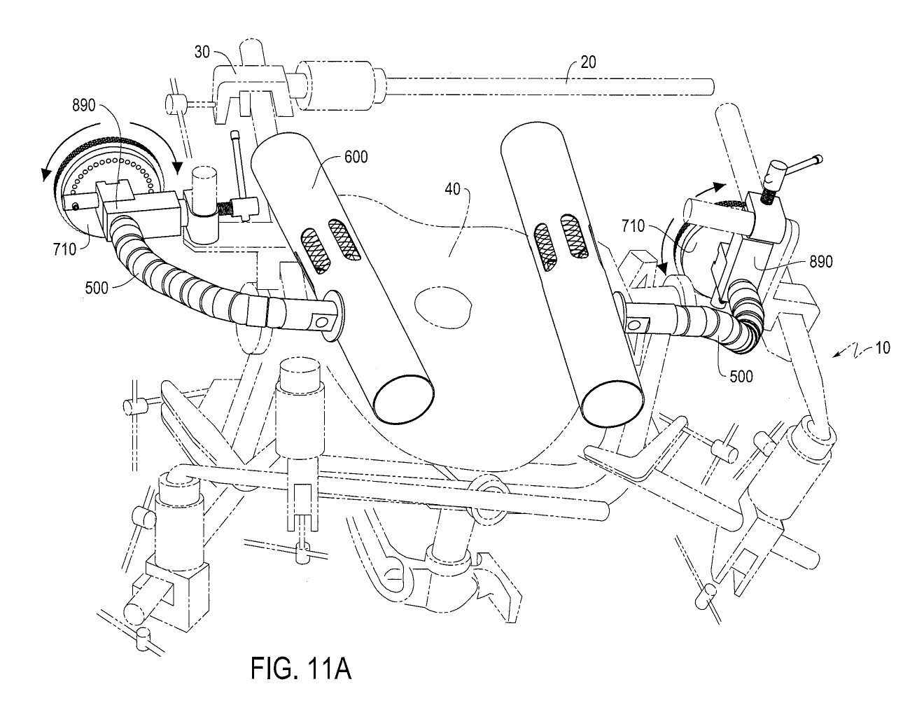

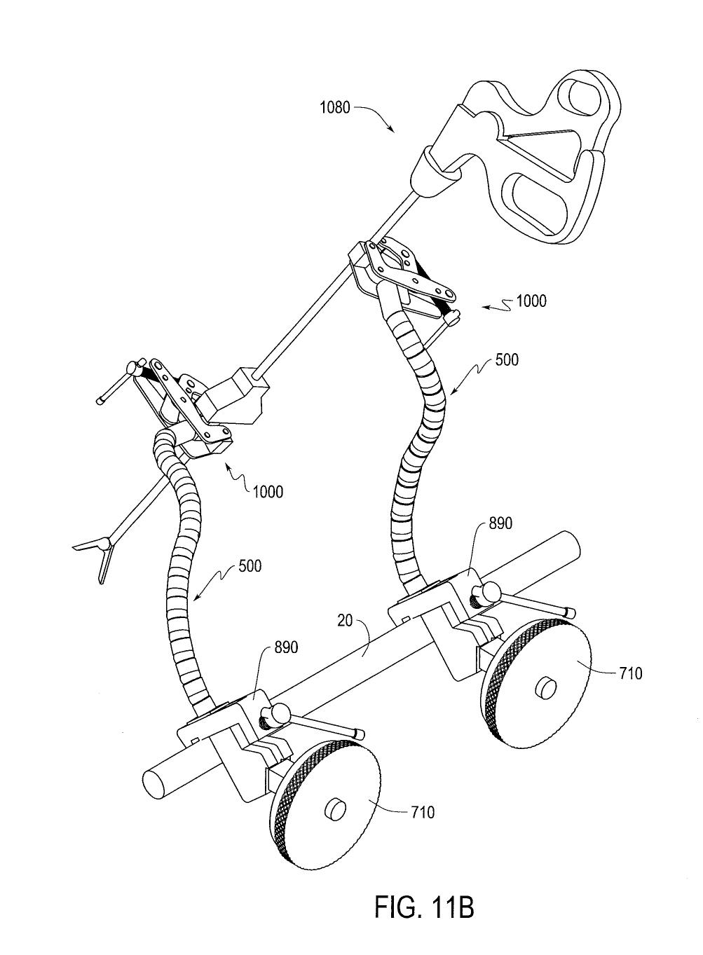

As shown in FIG. 11A, tension setting knob 710 may be connected to rigid framework system 10 such that rigid framework system 10 is disposed above and over surgical site 40. This may allow the medical instrument (for example, hand rest 600) to be disposed in an easy to access location for the surgeon during a surgical procedure. One or more attachment members 890 may securely connect a flexible arm 500 to a support bar 20. Additionally, connectors 30 may connect support bars 20 together to form rigid framework system 10. In some embodiments, two flexible arms 500 may be connected to a single medical instrument and to a single support bar 20 to provide increased stabilization for the medical instrument. For example, as shown in FIG. 11B, two flexible arms 20 are both connected to forceps 1080 through attachments 1000. The tension setting knob 710 connected to the first flexible arm 500 may provide the same or a different tension setting than the tension setting knob 710 connected to the second flexible arm 500.

The tension setting system 700 of the present disclosure can provide an easy to access and easy to manipulate system for a surgeon during a surgical procedure. Specifically, the surgeon may be able to readily identify the desired tension setting for a specific operation by identifying the tension setting locator 800 associated with that desired tension setting. The surgeon may then quickly set the tension setting to this locator when performing the operation. For example, a surgeon may know that he prefers first locator 810 when conducting delicate retraction. Therefore, the surgeon may set tension setting knob 710 to first locator 810 when doing such retraction of tissue. However, when the surgeon is then performing dissecting operations, the surgeon may know that he prefers third locator 830 and may quickly rotate tension setting knob 710 to third locator 830. Likewise, before the surgery begins, the surgeon or an assistant can preset each medical instrument at a desired setting for anticipated surgical tasks. This system eliminates and/or reduces the wasted time in finding the desired tension setting for each specific operation. Thus, the system of the present disclosure saves time and subsequent cost for the hospital and patient during surgical procedures, thereby reducing fatigue to the surgeon and reducing costs for the hospital and patient. Additionally, because the surgeon may quickly adjust tension setting system 700 himself, the number of nurses or surgical technicians, and thus the costs associated with each nurse/surgical technician, may be reduced during a surgical procedure. It is envisioned that tension setting system 100 may be used in such surgical procedures as, for example, craniotomy, general surgery, urological surgery, gynecological, and spinal surgery.

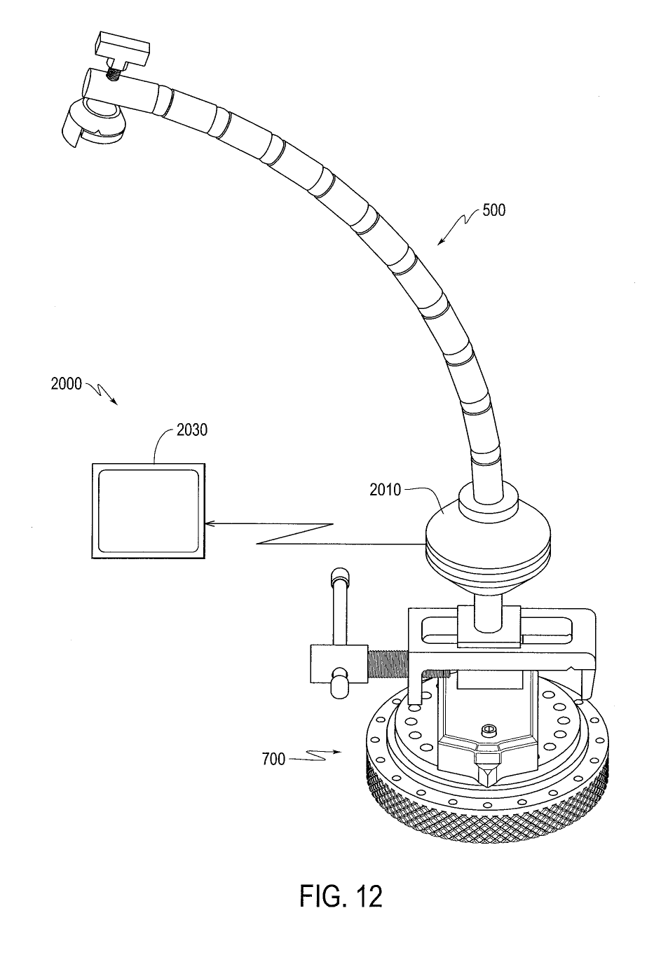

As shown in FIG. 12, the present disclosure is further directed to a tension setting quantification system 2000 which allows a user or the manufacturer of the tension setting system to determine the proper tension associated with each locator 800 on tension setting knob 710. The tension setting quantification system 2000 includes a load cell 2010 coupled to tension setting system 700 and flexible arm 500. Load cell 2010 may be directly and electrically connected to flexible cables 540 with a connecting cable (not shown), for example, a ribbon cable. It is further contemplated that one or more connecting and/or adaptor pieces may be used to connect load cell 2010 to flexible arm 500. In one embodiment, one or more arm segments 510 may be temporarily removed from flexible arm 500 so that load cell 2010 may be connected to flexible arm 500.

The load cell 2010 is configured to convert tension in tension cables 540 into a signal. An output display 2030 displays the signal to a user in a readable format. One or more resistors (not shown) may be provided in the circuitry between load cell 2010 and output display 2030 in order to produce the signal. In one embodiment, as the tension setting in tension cables 540 increases, for example by tightening tension cables 540 a predetermined amount, output display 2030 shows a reduction in voltage measured from load cell 2010. Furthermore, as the tension setting in tension cables 540 decreases, output display 2030 shows an increase in voltage measured from load cell 2010. Thus, a user is able to determine the relative tension in tension cables 540 when tension setting knob 710 is rotated to first locator 810, when tension setting knob 710 is rotated to second locator 820, etc. By viewing the signal on output display 2030, the user may determine if the tension in tension cables 540 associated with each locator 800 is proper (i.e., within a tolerance), or if the tension cables 540 need to be adjusted. For example, a user may determine that the tension in tension cables 540 are not correct, and the user may adjust the length of tension cables 540 (e.g., shorten tension cables 540).

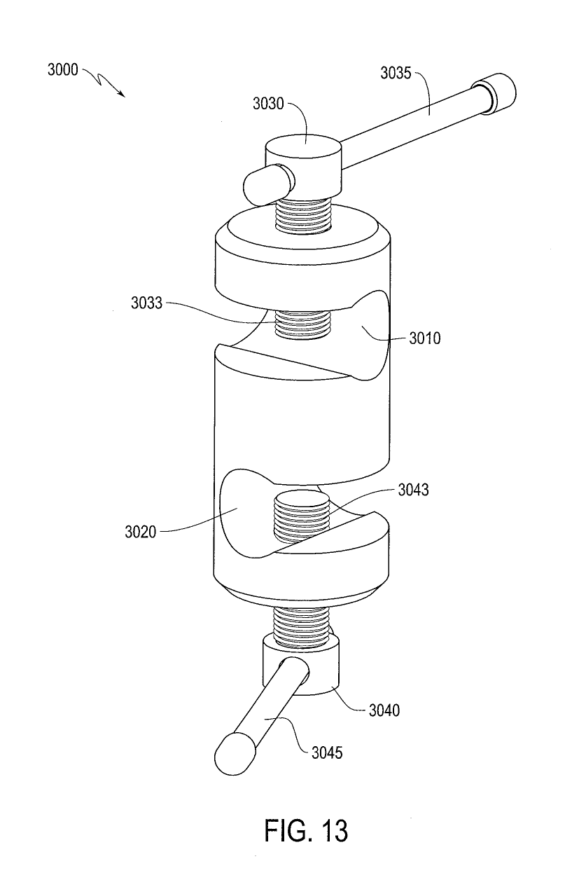

In some embodiments, one or more connectors 30 and/or attachment members 890 may include a complex connector 3000 including a first connector location 3010 and a second connector location 3020 (FIG. 12) that is oriented differently from the first connector location 3010. One or more support bars 20 may be disposed within first connector location 3010 and/or second connector location 3020 to form the rigid framework system 10. As shown in FIG. 13, first connector location 3010 and second connector location 3020 each include a depression formed within complex connector 3000. In some embodiments, the depressions may have a substantially cylindrical shape and sized to correspond to a support bar 20. Furthermore, complex connector 3000 may include a first locking mechanism 3030 to secure one or more support bars 20 within first connector location 3010. The first locking mechanism 3030 may include a screw member 3033 configured to be screwed into and out of first connector location 3010 by rotating member 3035 to selectively lock a support bar 20 within first connector location 3010. Similarly, complex connector 3000 may include a second locking mechanism 3040 to secure one or more support bars 20 within second connector location 3020. The second locking mechanism 3040 may include a screw member 3043 configured to be screwed into and out of second connector location 3020 by rotating member 3045 to selectively lock a support bar 20 within second connector location 3020.

The complex connector 3000 allows a first support bar 20 to be disposed within first connector location 3010 in a first orientation so that the first support bar 20 extends in a first direction, and may allow a second support bar 20 to be disposed within second connector location 3020 in a second orientation so that the second support bar 20 extends in a second direction that is different from the first direction. The first direction may be substantially perpendicular to the second direction. However, it is further contemplated that the first direction may be disposed at various angles to the second direction, e.g., 10 degrees, 20 degrees, 45 degrees, 85 degrees, 150 degrees, etc. This complex connector 30 allows the rigid framework system 10 to be easily assembled with varying flexibility in the location of each support bar 20. Additionally, during for example, laparoscopic procedures, complex connectors 3000 may connect multiple support bars 20 to form rigid framework system 10 such that flexible arms 500 may be directly attached to the support bars 20. This allows the surgical instruments to be disposed above and over surgical site 40 and in an easy to access location for the surgeons.

It will be apparent to those skilled in the art that various modifications and variations can be made to the system of the present disclosure. It is intended that this disclosure and examples herein be considered as exemplary only, with a true scope of the disclosure being indicated by the following claims and their equivalents.

What is claimed is:

1. An implement holding device for use in surgeries, the device comprising:
a flexible arm defining an inner lumen and having a portion that is configured to be coupled to a medical instrument;
a tension cable disposed within the inner lumen and configured to adjust the rigidity of the flexible arm; and
a tension setting dial coupled to the tension cable and configured to be manipulated to adjust tension of the tension cable, the tension setting dial including a plurality of discrete tension setting locators, wherein
each tension setting locator is configured to be engaged when the tension setting dial is in a position corresponding to the specific tension setting locator,
each tension setting locator corresponds to a different cable tension force such that manipulating the tension setting dial to a position corresponding to one of the tension setting locators adjusts the tension of the tension cable to a specific cable tension force, and
changing the position of the tension setting dial is effective to change the rigidity of the flexible arm.

2. The implement holding device of claim 1, wherein each tension setting locator is pre-calibrated to correspond to its respective cable tension force.

3. The implement holding device of claim 1, wherein the tension setting dial includes different markings adjacent to each tension setting locator that are indicative of the corresponding cable tension force.

4. The implement holding device of claim 1, further comprising a position indicator with an indicator end portion, wherein the tension setting dial moves relative to the indicator end portion when the tension setting dial is adjusted, and the position indicator engages one of the tension setting locators when the tension setting dial is positioned to align the indicator end portion with the tension setting locator.

5. The implement holding device of claim 4, wherein each of the plurality of tension setting locators comprise an indent on a surface of the tension setting dial.

6. The implement holding device of claim 5, wherein the position indicator includes a ball configured to be disposed in the indent of each tension setting locator when the position of the tension setting dial is aligned with the respective tension setting locator.

7. The implement holding device of claim 6, wherein the position indicator further includes a spring that is configured to urge the ball into the indent.

8. The implement holding device of claim 7, wherein the position indicator further includes a rod configured to hold the spring and the ball so that the ball is disposed partly outside of the rod.

9. The implement holding device of claim 1, wherein the tension setting dial further includes a stop so that a user cannot adjust the position of the tension setting dial to be beyond the stop.

10. The implement holding device of claim 1, wherein the tension setting dial is substantially circular and the position of the tension setting dial is adjusted by rotating the tension setting dial.

11. The implement holding device of claim 10, wherein the tension setting dial further includes a knurled surface on an outer perimeter surface of the tension setting dial.

12. The implement holding device of claim 10, wherein the tension setting locators are arranged on a side of the tension setting dial, each tension setting locator being configured to be engaged when the tension setting dial is rotated to a position corresponding to the specific tension setting locator.

13. The implement holding device of claim 1, wherein adjusting the position of the tension setting dial causes the tension cable to twist within the inner lumen of the flexible arm.

14. The implement holding device of claim 1, wherein the flexible arm comprises a plurality of segments each cooperating with an adjacent segment and allowing the flexible arm to be bent into different shapes by a user.

15. The implement holding device of claim 14, wherein the flexible arm further includes a plurality of ball joints that separates adjacent segments of the plurality of segments.

16. The implement holding device of claim 1, wherein, when the flexible arm is relatively more rigid, a greater force is required to move the flexible arm.

17. The implement holding device of claim 1, wherein an end portion of the flexible arm includes an attachment member that is configured to secure the implement holding device to a rigid framework.

18. The implement holding device of claim 17, wherein the rigid framework is configured to be secured to an operating table.

19. The implement holding device of claim 1, wherein the medical instrument includes an instrument selected from the group consisting of: a retractor blade, a dissector, forceps, an endo scope, a rotary cutter, a drill, an aspirator, an ultrasonic probe, a suction tube, and a surgical hand rest.

20. The implement holding device of claim 1, wherein the flexible arm further includes a removable attachment configured to secure the medical instrument to the flexible arm.

21. The implement holding device of claim 20, wherein the removable attachment is customized for the medical instrument.

22. A method of adjusting tension in the implement holding device of claim 1, the method comprising:
rotating the tension setting dial from a first tension setting locator to a second tension setting locator to adjust the tension of the tension cable;
manipulating the position of the flexible arm; and
applying a downward force on the flexible arm such that the flexible arm provides sufficient tension and pressure against the downward force.

23. The method of claim 22, further comprising rotating the tension setting dial to a third tension setting locator.

24. A tension setting quantification system comprising:
a load cell coupled to the tension cable of the implement holding device of claim 1, such that the load cell is configured to convert the tension in the tension cable into a signal; and
an output display configured to display the signal to a user.

25. A method of quantifying tension in the tension setting quantification system of claim 24, the method comprising:
adjusting the tension in the tension cable by rotating the tension setting dial to a tension setting locator; and
adjusting the length of the tension cable if the output display produces a signal that does not correspond to a predetermined cable tension associated with the tension setting locator.

26. A tension setting quantification system comprising:
a load cell coupled to a tension cable such that the load cell is configured to convert tension in the tension cable into a signal; and
an output display configured to display the signal to a user, wherein
the tension cable is disposed within an inner lumen of a flexible arm and is configured to adjust the rigidity of the flexible arm.

27. A method of quantifying tension in the tension setting quantification system of claim 26, the method comprising:
adjusting tension in the tension cable by tightening the tension cable a predetermined amount; and
adjusting the length of the tension cable if the output display produces a signal that does not correspond to a predetermined cable tension associated with the predetermined tightening.

28. An implement holding device, the device comprising:
a flexible arm defining an inner lumen;
a tension cable disposed within the inner lumen and configured to adjust the rigidity of the flexible arm; and
a tension setting dial coupled to the tension cable and configured to be manipulated to adjust tension of the tension cable, the tension setting dial including a plurality of discrete tension setting locators, wherein
each tension setting locator is configured to be engaged when the tension setting dial is in a position corresponding to the specific tension setting locator,
each tension setting locator corresponds to a different cable tension force such that manipulating the tension setting dial to a position corresponding to one of the tension setting locators adjusts the tension of the tension cable to a specific cable tension force, and
changing the position of the tension setting dial is effective to change the rigidity of the flexible arm.
